(19) United States
(12) Patent Application Publication
(10) Pub. No.: US 2008/0214931 A1
(43) Pub. Date: Sep. 4, 2008

(54) Method and System for Guiding a Probe in a Patient for a Medical Procedure

(76) Inventor: Timm Dickfeld, Baltimore, MD (US)

Correspondence Address: EVANS & MOLINELLI, PLLC, U.S. POST OFFICE BOX 7024, FAIRFAX STATION, VA, 22039, US

(21) Appl. No.: 11/993,963

(22) PCT Filed: Jun. 26, 2006
(86) PCT No.: PCT/US06/24708

§ 371 Date: Dec. 26, 2007

Related U.S. Application Data
(60) Provisional application No. 60/694,781, filed on Jun. 28, 2005.

(51) Int. Cl. A61B 5/05

(52) U.S. Cl. 600/424

(57) ABSTRACT

Techniques for using a probe in a patient for a medical procedure based on different modes of operating sensor systems than currently a used includes receiving first volume data based on a first mode of operating a first sensing system. In addition, different second volume data is received based on a second mode of operating a second sensing system. Probe position data is also received. The probe position data indicates a position of a probe inserted in the particular patient based on a probe positioning system independent of the first sensing system and the second sensing system. Probe position relative to the first volume data and the second volume data is determined. The operation of the probe is guided based on the probe position relative to the first volume data and the second volume data.

### CROSS-REFERENCE TO RELATED APPLICATIONS

This application claims benefit of Provisional Appln. 60/694,781, filed Jun. 28, 2005, the entire contents of which are hereby incorporated by reference as if fully set forth herein, under 35 U.S.C. §119(e).

### BACKGROUND OF THE INVENTION

1. Field of the Invention

The present invention relates to using volume imaging of a patient to guide the use of a probe inserted into a patient to provide medical diagnostic or therapeutic care to that patient, and in particular to the use of volume imaging involving nuclear magnetic resonance imaging (MRI), computer tomography (CT) and Positron Emission Tomography (PET) or multiple different modes of volume imaging or both to guide ablation operations during interventional electrophysiological procedures. As used herein, volume imaging refers to any means for obtaining images of the interior of a patient and is not intended to imply any limit in spatial resolution, dynamic range or degree of tissue differentiation.

2. Description of the Related Art

Interventional electrophysiological procedures can diagnose and cure a wide variety of heart arrhythmias involving the atria and ventricles. This is mostly achieved by identifying an area or structure within the heart (or the adjacent vessels) that is involved in initiating or sustaining the arrhythmia. Multiple, mostly catheter-based procedures have been developed to define and destroy those arrhythmia-causing areas or structures using different kind of energy (including, but not limited to, radiofrequency, laser, ultrasound, cooling, and injection of cell toxins) in a process called ablation. Ablation lesions formed in the heart tissue by the ablation process block the initiation, modulation or propagation of spurious electrical signals that lead to arrhythmia.

The identification of the target areas/structures has traditionally been performed by using electrical signals detected by the electrophysiological catheter. Over the last decade a paradigm shift has occurred in which treatment strategies involve anatomic rather than electrical considerations. These procedures require placing an ablation lesion at an exact anatomic location in the heart (e.g., at the cavotricuspid isthmus or pulmonary vein orifice). Such anatomic-based ablations successfully treat atrial flutter, atrial fibrillation, and non-idiopathic ventricular tachycardia, VT.

Because Roentgen (x-ray) fluoroscopy has very limited abilities to visualize arrhythmia-causing structures, electro-anatomical mapping systems have become increasingly important. These mapping systems are able to determine the catheter tip location in real-time using a variety of different technologies (like ultrasound, electrical fields, electromagnetic fields) and to display the location on a cartoon image of the endocardium—the inside shell of the heart (Gepstein L, Hayam G, Ben-Haim S A. A novel method for nonfluoroscopic catheter-based electroanatomical mapping of the heart. In vitro and in vivo accuracy results, Circulation. v18; #95(6), pp 1611-22, Mar. 1997; de Groot N M, Bootsma M, van der Velde E T, Schalij M J, Three-dimensional catheter positioning during radiofrequency ablation in patients: first application of a real-time position management system, J Cardiovasc Electrophysiol, v11 (#11), pp 1183-92, November 2000). The cartoon image is based on either contact points mapped by the catheter tip or generic models of the heart chambers.

Given the limited anatomic resolution of these cartoon images, a newer generation of mapping systems are expected to replace the inner shell reconstruction from multiple catheter locations by an inner shell extracted from computer tomography of low intensity Roentgen rays (CT) or nuclear magnetic resonance (NMR) imaging (MRI) images that are presented to a doctor as three-dimensional surface renderings (Dickfeld T, Calkins H, Zviman M, Kato R, Meininger G, Lickfett L, Berger R, Halperin H, Solomon S., “Anatomical Stereotactic Catheter Ablation on Three-Dimensional Magnetic Resonance Images in Real Time,” Circulation, v108, pp 2407-2413, 2003). Such a new generation mapping system is described in more detail in a later section.

Even the newest generation of these mapping systems that are used to navigate the catheter tip to a certain endocardial structure are limited in several ways. The systems display only the blood-endocardium interface, but cannot supply any anatomic information about the myocardial structures (within the walls of the heart chambers). Besides anatomic information, these systems do not supply any other information, such as histology or biological or chemical processes. These limits have become evident in using purely endocardial anatomical guidance (and its electrical surrogates) for complex ablation procedures to treat atrial fibrillation and non-idiopathic ventricular tachyeardia (VT). Because the exact location of successful ablation sites is not purely defined by endocardial anatomy, long procedure times to ablate large areas of the heart walls have been implemented with only moderate treatment success. Additional strategies now include considering electrical signal properties at the catheter tip in combination with anatomical procedures (Nademanee K, McKenzie J, Kosar E, Schwab M, Sunsaneewitayakul B, Vasavakul T, Khunnawat C, Ngarmukos T “A new approach for catheter ablation of atrial fibrillation: mapping of the electrophysiologic substrate,” J Am Coll Cardiol. 2; v43, #11, pp 2044-53, June 2004).

Based on the foregoing, there is a clear need for techniques to guide ablation with an invasive electrophysiological catheter that does not suffer the limitations of prior art approaches. In general, there is a need for techniques to guide a probe for medical procedures inside a patient using information in addition to that available at the probe and from a single surface anatomical rendering based on a single mode of operating sensing system like CT and MRI.

### SUMMARY OF THE INVENTION

Techniques are provided for using a probe in a patient for a medical procedure based on different modes of operating sensor systems than currently used. In some embodiments, multiple modes are used. In other embodiments, a single mode of operating the sensing system is employed but different structures are derived from the single mode, such as molecular processes derived from a single mode of operating a Positron Emission Tomography, PET, sensing system or multiple boundaries rendered based on a HRI or CT sensing system, or a single boundary generated by a new imaging technique, such a PET and other emerging nuclear medicine techniques, like single photon emission computed tomography (SPECT).

In one set of embodiments, a method includes receiving first volume data based on measurements of a particular volume in a particular patient from a first mode of operating a first sensing system. In addition, different second volume data is received based on measurements of the particular volume in the particular patient from a second mode of operating a second sensing system (which may be a different mode of the same sensing system as the first). Probe position data is also received. The probe position data indicates a position of a probe inserted in the particular patient based on a probe positioning system independent of the first sensing system and the second sensing system. Probe position relative to the first volume data and the second volume data is determined. The operation of the probe is guided based on the probe position relative to the first volume data and the second volume data.

In some embodiments of this set, the step of operating the probe includes at least one of changing a position of the probe, activating the probe to sample nearby tissue, activating the probe to apply therapy to nearby tissue, and presenting a three dimensional graphical rendering of the first volume data in relation to the second volume data and the probe position data for viewing by a human medical care giver who determines how to operate the probe based on the three dimensional graphical rendering.

In some embodiments for using an interventional electrophysiological catheter in a patient for treating arrhythmia, the method includes receiving first volume data, different second volume data and catheter position data. The first volume data indicates the endocardial surface or myocardial structures of the cardiac chambers in a particular patient based on first measurements from a first sensing system, such as an MRI system. The second volume data indicates at least one of myocardial structure and myocardial process in a wall of the chamber of the heart of the particular patient based on different second measurements from a second sensing system, such as a PET system. The second system can be the same as the first sensing system using a different variable component of the system, such as an MRI with a different target or contrasting agent, a CT scan acquired at different time point after contrast injection or a PET with a different tracer compound. The catheter position data indicates a position of an interventional electrophysiological catheter inserted in the chamber of the heart of the particular patient based on an electrophysiological catheter positioning system, such as an electromagnetic sensing system, independent of the first sensing system and the second sensing system. The catheter position relative to the endocardial surface and the myocardial structure is determined. The catheter is operated based on the catheter position relative to the endocardial surface and the myocardial structure to perform at least one of moving the catheter and ablating tissue in a vicinity of a tip of the electrophysiological catheter.

In another set of embodiments of the invention, a method includes receiving volume data based on measurements of a particular patient from a Positron Emission Tomography (PET) system and receiving probe position data. Probe position is determined relative to the volume data. The probe is operated based on the probe position relative to the volume data.

In another set of embodiments, a method includes receiving volume data and probe position data. The volume data is based on measurements of a particular patient from a particular sensing system. The probe position data indicates a position of a probe inserted in the particular patient based on a probe positioning system independent of the particular sensing system. Multiple different boundaries representing corresponding different anatomical features are determined in the volume data. A probe position relative to the different boundaries is determined. The probe is operated based on the probe position relative to the different boundaries.

In other sets of embodiments, the techniques include a system, a computer-readable medium, and an apparatus that implement some or all the steps of the above methods.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1A is a block diagram that illustrates a probe ablation system;

FIG. 1B is a block diagram that illustrates a volumetric imaging system;

FIG. 2A is a block diagram illustrates corresponding points on two 3-D renderings, one constructed from electro-anatomic mapping and the other from computer tomography (CT) images;

FIG. 2B, FIG. 2C, FIG. 2D, FIG. 2E are block diagrams that indicate overlapping renderings after registration using four different registration techniques;

FIG. 3A is a set of images that show five views of an endocardial surface of the heart based on electro-anatomical mapping;

FIG. 3B is a set of images that show the same five views of an endocardial surface of the heart based on positron-emission tomography mapping;

FIG. 4 is a block diagram that illustrates a system for guiding a probe in a patient, according to an embodiment;

FIG. 5 is a flow diagram that illustrates at a high level a method for operating a probe based on one or more different modes of one or more sensing systems, according to an embodiment; and

FIG. 6 is a block diagram that illustrates a computer system upon which an embodiment of the invention may be implemented.

### DETAILED DESCRIPTION

Techniques are described for guiding the operation of a probe, such as an interventional electrophysiological catheter, in a patient based on different modes of operating sensor systems than currently used. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are descried below in the context of interventional electrophysiological procedures to treat arrhythmia. However, the invention is not limited to this context. In other embodiments other probes are used in other medical procedures to diagnose or treat other conditions in a patient. Furthermore, measurement modalities are described in the context of a few sensing systems; however, it is anticipated that new sensing systems yet to be developed will be utilized in some embodiments.

For example, in various other embodiments, the same intervention can be used to treat a wide variety of pathophysiological processes with interventional procedures. In cardiology, a probe is operated in a patient to introduce gene therapy, inject stem cells, perform interventional (re)vascularization or initiate angiogenesis and perform biopsies to diagnose or treat a variety of conditions such as ischemia, scar formation, abnormal metabolism, electrical activity or neuronal input. In hematology-oncology, a probe is operated in a patient to obtain targeted destruction of malignant cells and is important for the treatment of cancer, such as cancers in liver, lung, kidney, pancreas, brain, breast, lymphatic system, bone marrow, and metastasis or sub-selective cell groups identified with specialized molecular and metabolic imaging. In endocrinology, a probe is operated in a patient to introduce gene therapy, inject stem cells, and perform biopsies to diagnose or treat a variety of conditions such as endocrinal hypo-activity and hyper-activity in organs such as thyroid gland, adrenal gland, pancreas etc. In neurology, a probe is operated to target areas with increased or decreased amount of transmitters or with structural changes, or with abnormal growth, or with changes in metabolic behavior. In an expanding interventional neurological field, a probe is used in patients to enhance, manipulate or destroy neuronal inputs to other organ systems. In pulmonary medicine, nephrology, urology, gynecology, and interventional radiology, a probe is operated to target areas with abnormal growth, different surface receptors, different gene expression and different surface molecules and to achieve therapeutic modulation of these factors as well as the placement of electrical or nanotechnology devices with therapeutic intentions in mind. In minimal invasive surgery, a probe is used to guide the removal of abundant, malignant, diseased or constrictive tissue and allow for therapeutic manipulation.

### 1. Structural Overview

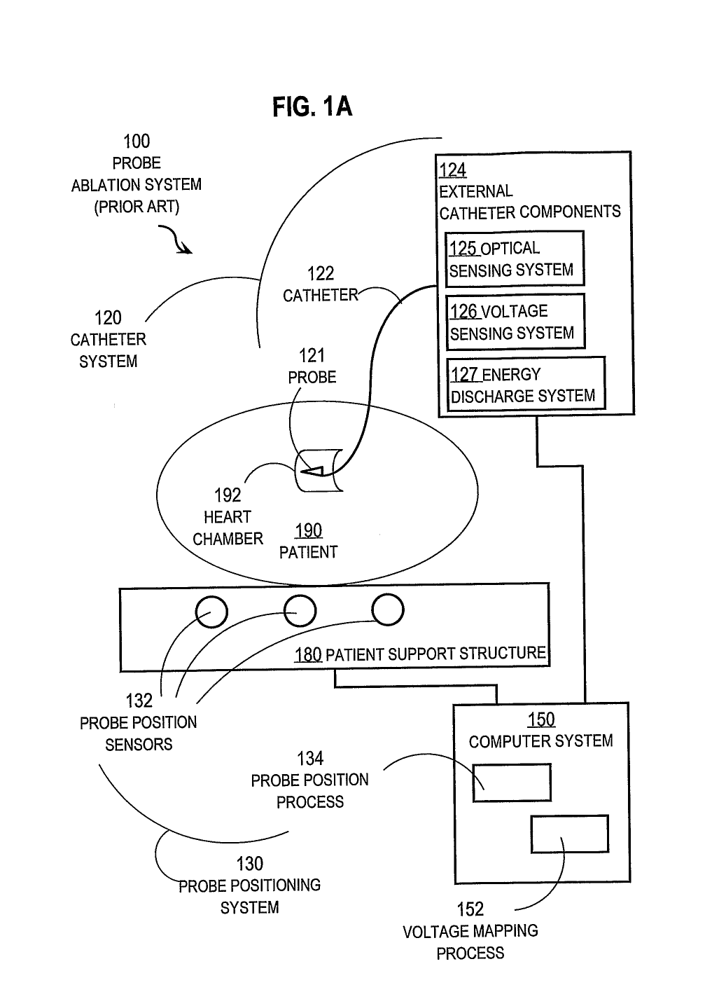

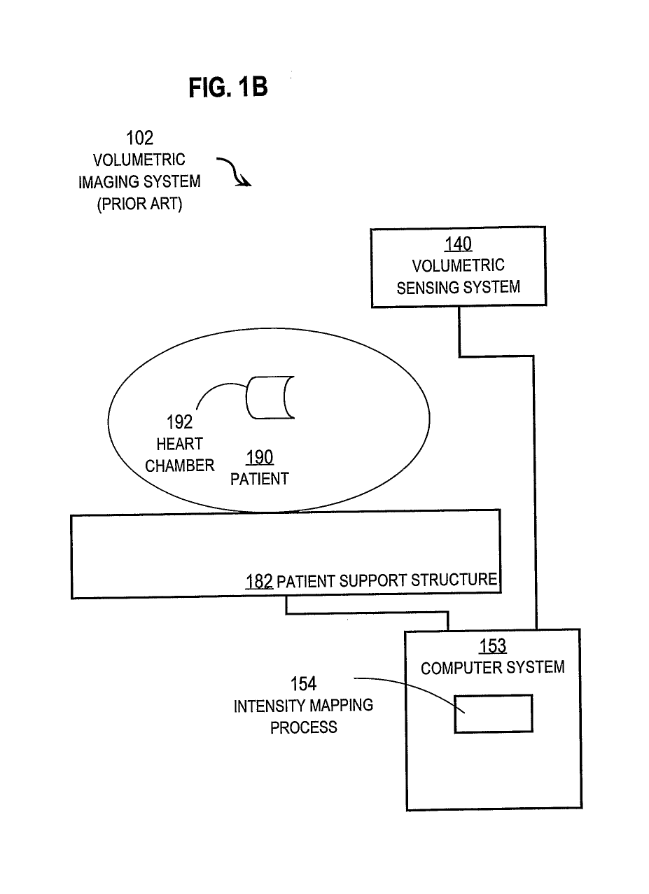

FIG. 1A is a block diagram that illustrates a probe ablation system 100, such as used in previous approaches and anticipated in future approaches. The probe ablation system includes a patient support structure 180, a catheter system 120 and a computer system 150. Also depicted in FIG. 1A is a patient 190 having a heart chamber 192, which is a target of a probe for the ablation system. However, the patient 190 and heart chamber 192 are not part of the probe ablation system 100.

The catheter system 120 includes a probe 121, a catheter 122, and external catheter components 124. The probe is any device used to measure or treat tissue structure or function in a patient. For example, the probe is a tip of a catheter with a voltage sensor, an optical with or without an imaging device, and an energy discharge device to ablate tissue upon command. The catheter 122 is any narrow, elongated structure constructed to be introduced inside the body of a patient, including tubes that follow blood vessels or other body lumen and needles that penetrate flesh. The catheter is constructed to pass signals or therapeutic agents or both between the probe 121 and external catheter components 124.

The external catheter components 124 include one or more sensing systems, such as optical sensing system 125 and voltage sensing system 126. Optical sensing system 125 receives and processes optical signals transferred from probe 121 through catheter 122, such as an image of tissue directly in front of the probe illuminated by natural or artificial light. Voltage sensing system 126 receives and processes electrical signals proportional to voltage detected at the tip of probe 121 and transmitted through catheter 122. Signals indicative of electrical pathways active in arrhythmia, for example, are determined by voltage sensing system 126. The external catheter components also include zero or more agent delivery systems, such as energy discharge system 127. For example, when the probe is positioned at a location in heart tissue where a lesion is desired to influence electrical conduction important to treat arrhythmia, a signal is sent to the probe to discharge an electric current sufficient to form such a lesion.

To guide the probe 121 and catheter 122 through the patient 190 to a desired location in a target, such as heart chamber 192, the probe positioning system 130 is employed. In the illustrated system, probe positioning system 130 includes probe position sensors 132 embedded in patient support structure 180, and probe position process 134, executing on computer system 150. Based on the measurements made by sensors 132, the process 134 determines the three-dimensional location of probe 121.

In the illustrated system, computer system 150 includes a voltage mapping process 152. The voltage mapping process 152 associates the voltage detected at probe 121 with a location of the probe from probe positioning system 130 and maps the distribution of voltage along a target, such as along an endocardial surface of the heart chamber 192, as the probe moves along that surface. The measured distribution of voltage can then be displayed using well-known methods for surface rendering to produce a three-dimensional rendering of voltage. In previous approaches, ablation strategies were formulated and executed based on the 3-D mapping of voltages alone. Even without mapping voltage, a coarse volume of the inside of a chamber can be determined by moving the probe within the chamber and recording probe locations when touching a chamber wall.

FIG. 1B is a block diagram that illustrates a volumetric imaging system 102, such as a MRI or CT imaging system. The volumetric imaging system includes a patient support structure 182, a sensing system 140, and a computer system 153. Also depicted in FIG. 1B is a patient 190 having a heart chamber 192, which is a target of the volumetric imaging system 102. However, the patient 190 and heart chamber 192 are not part of the volumetric imaging system 102.

The volumetric sensing system 140 emits or detects, or both, wave energy transmitted through patient 190, such as high frequency acoustic waves and electromagnetic waves, as is well known in the art of medical imaging. These measurements are used by an intensity mapping process 154 in computer system 153 to generate one or more images of intensity differences detected inside the patient 190. Multiple images can be combined to form a three-dimensional rendering of one or more regions of practically homogeneous intensity. An analyst associates the tissue shape and position as anatomical features.

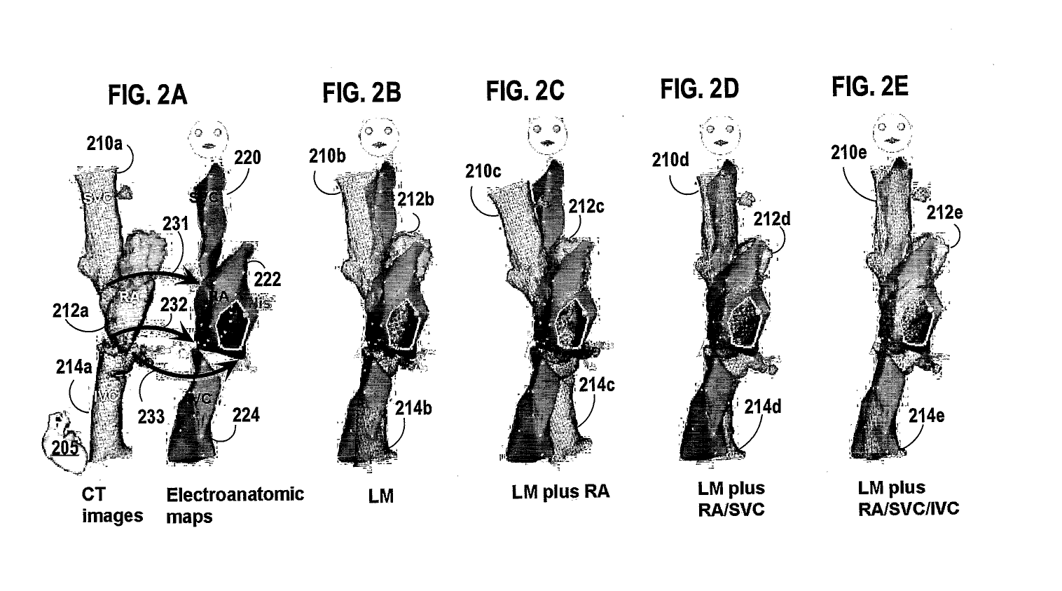

FIG. 2A is a block diagram illustrates corresponding points on two 3-D renderings of an animal heart, one constructed from electro-anatomic mapping and the other from CT images. The position of the structures in the animal heart is indicated by a black dot atop the heart 205 depicted in FIG. 2A. A 3-D rendering based on CT scans (such as from intensity mapping process 154) appears on the left. An endocardial surface based on electro-anatomical mapping (such as from voltage mapping process 152), appears on the right. FIG. 2B, FIG. 2C, FIG. 2D, FIG. 2E are block diagrams that indicate overlapping renderings after registration using four different registration techniques. FIG. 2A and following figures FIG. 2B, FIG. 2C, FIG. 2D and FIG. 2E are based on figures from Duong, J., Calkins H, Solomon S, Lai S, Dalal D, Lardo A, Brem E, Preiss A, Berger R D, Halperin H, Dickfeld T. Integrated electroanatomic mapping with three-dimensional computed tomographic images for real-time guided ablations,” Circulation, v113, pp 186-194, 2006, hereinafter Dong,

In some approaches, such as described in Dong, the extra fine resolution and tissue variation information available from CT are used along with an interventional electrophysiological probe (such as probe 121) to treat arrhythmia. For such apporaches, the two 3-D renderings must be registered so that corresponding points on both renderings occupy the same location. This registration process then positions the probe with respect to the CT rendering. Any registration method may be used.

Each of the renderings include a superior vena cava (SVC 210a in the CT rendering, and SVC 220 in the electro-anatomic rendering), a right atrium (RA 212a in the CT rendering, and RA 222 in the electro-anatomic rendering), and an inferior vena cava (IVC 214a in the CT rendering, and IVC 224 in the electro-anatomic rendering).

In Dong, one step of the registration process includes identifying landmarks evident in each of the images. For example, in Dong, three landmarks are used to register the two renderings: the coronary sinus (CS) ostium, the SVC-RA junction and the IVC-RA junction. The landmark registration is accomplished by orienting one of the renderings (e.g., the CT rendering) so as to minimize the distance between each of the three landmarks on the CT rendering with the corresponding landmarks on the electro-anatomic rendering. The arrow 231 points from the SVC-RA junction on the CT rendering to the SVC-RA junction on the electro-anatomic rendering. Similarly, the arrow 232 points from the IVC-RA junction on the CT rendering to the IVC-RA junction on the electro-anatomic rendering. The arrow 233 points from the CS ostium on the CT rendering to the CS ostium on the electro-anatomic rendering.

FIG. 2B depicts the relative positions of the two renderings after the landmark registration. The locations of the SVC, RA and IVC of the electro-anatomic rendering are the same as in FIG. 2A. The CT rendering is rotated relative to FIG. 2A with the SVC 210b, RA 213b and IVC 214b as depicted.

As described in Dong, surface registration is sometimes combined with landmark registration. Surface registration minimizes an average distance between endocardial surfaces in the electro-anatomical rendering and the CT rendering for selected portions of those renderings. FIG. 2C depicts the relative positions of the two renderings after the landmark registration combined with surface registration for the RA regions only. The locations of the SVC, RA and IVC of the electro-anatomic rendering are the same as in FIG. 2A. The CT rendering is further rotated relative to FIG. 2A with the SVC 210c, RA 213c and IVC 214c as depicted. FIG. 2D depicts the relative positions of the two renderings after the landmark registration combined with surface registration for both the RA and SVC regions. The CT rendering better overlaps the electro-anatomic rendering relative to FIG. 2A, with the SVC 210d, RA 213d and IVC 214d as depicted. FIG. 2E depicts the relative positions of the two renderings after the landmark registration combined with surface registration for the RA SVC and ISVC regions. The CT rendering overlaps the electro-anatomic rendering similarly to FIG. 2D, with the SVC 210e, RA 213e and IVC 214e as depicted.

### 2. Functional Overview

According to various embodiments of the invention, new measurement modalities or combinations of measuring modalities are employed with probe positioning systems to guide a probe to a target site for diagnosis or treatment. For example, in some embodiments, both a rendering based on MRI and a rendering based on CT are used with the probe mapping system to guide the probe to a target site. In some embodiments, a rendering based on positron-emission tomography (PET) is used with the probe mapping system to guide the probe to a target site.

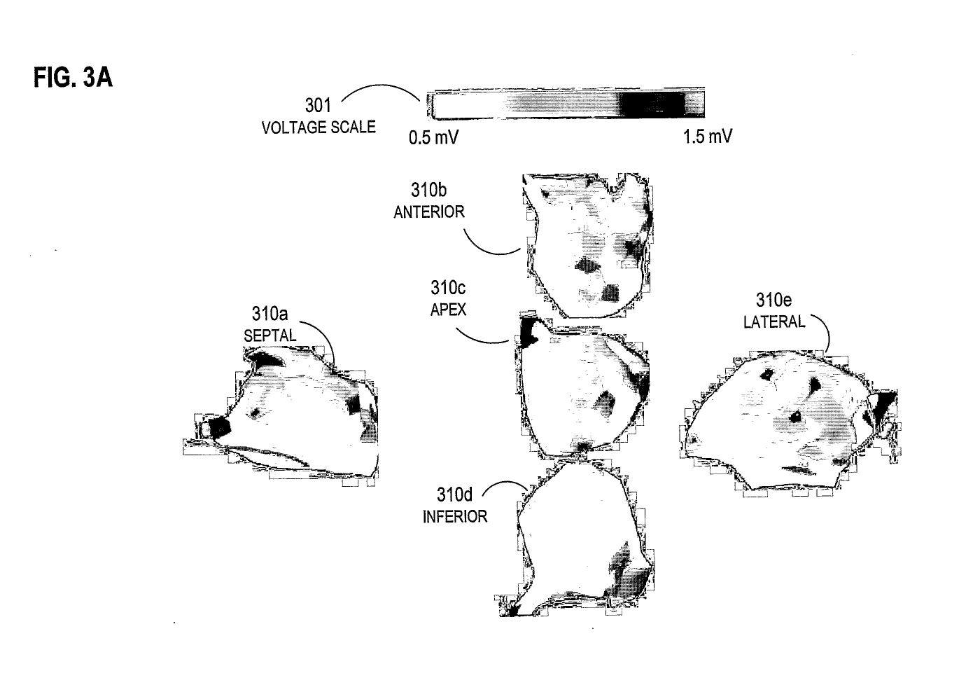

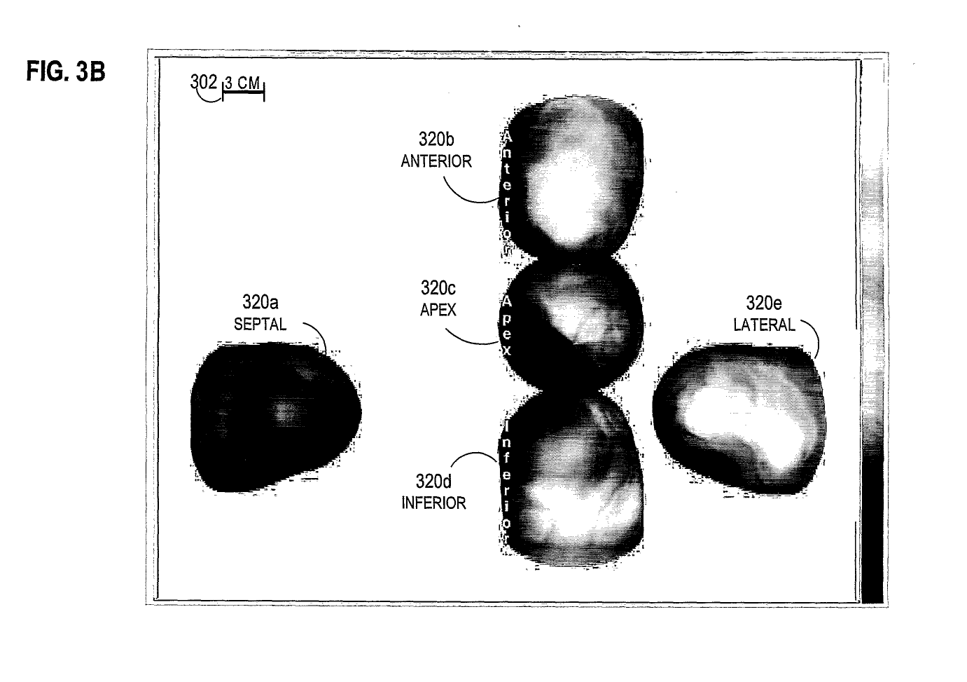

FIG. 3A is a set of images that show five views of an endocardial surface of the heart based on electro-anatomical mapping. Voltage is mapped as a function of probe position along the endocardial surface of a heart. FIG. 3A includes a voltage scale 301, a septal view 310a, an anterior view 310b, an apex view 310c, an inferior view 310d and a lateral view 310e.

FIG. 3B is a set of images that show the same five views of the endocardial surface of the heart based on positron-emission tomography mapping. PET tracer uptake is mapped as a function of position along the endocardial surface of the same heart as mapped in FIG. 3A. FIG. 3B includes a distance scale 302, a septal view 320a, an anterior view 320b, an apex view 320c, an inferior view 320d and a lateral view 320e.

PET allows noninvasive assessment of regional blood flow, function and metabolism using physiological substrates prepared with positron-emitting isotopes, e.g, carbon, oxygen, nitrogen and fluorine. When the high-energy positron is emitted from a nucleus, it travels a short distance and collides with an electron. The result is complete annihilation of both the positron and the electron and conversion of the combined mass to energy in the form of electromagnetic radiation (two gamma rays, 511 keV each). Because the gamma rays are perfectly collinear (discharged at 180 degrees to each other) and travel in opposite directions, the PET detectors are programmed to register only events with temporal coincidence of photons that strike directly at opposing detectors. This results in improved spatial (4-6 mm) and temporal resolution. Moreover, the PET system is more sensitive (higher count rate) than a spectral camera system and affords the possibility of attenuation correction. As a consequence, PET offers the possibility for quantification of tracer concentration in absolute units.

As can be seen by comparing FIG. 3A and FIG. 3B, the PET data (FIG. 3B) shows higher spatial resolution than the probe voltage measurements (FIG. 3A). Furthermore, the PET data (FIG. 3B) shows different spatial variations. For example, the PET lateral view 320e shows a different variation than the voltage lateral view 310e.

### 3. Probe Guidance System

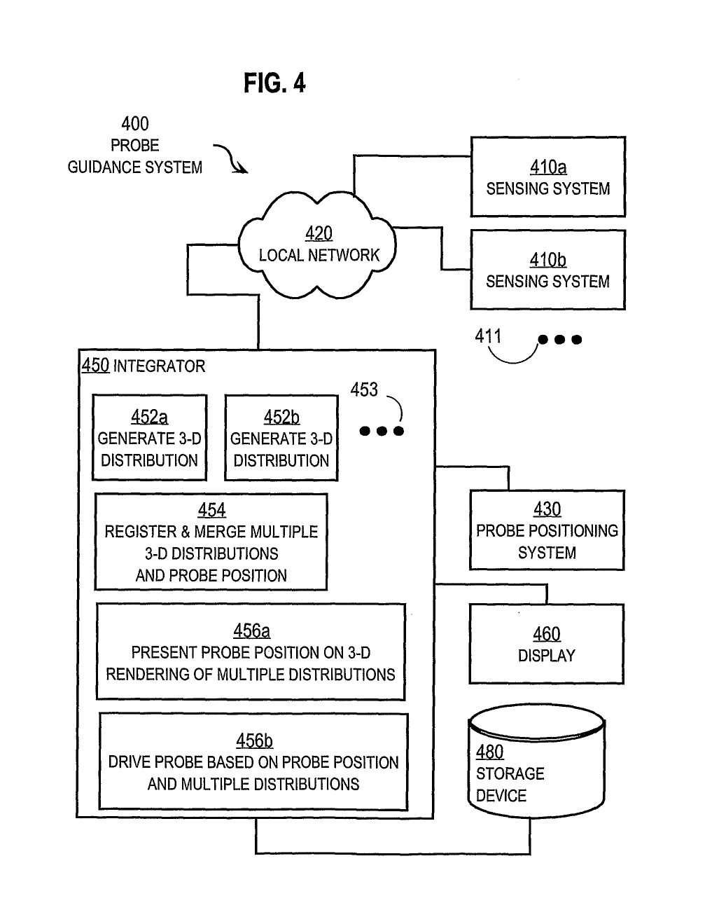

FIG. 4 is a block diagram that illustrates a system 400 for guiding a probe in a patient, according to an embodiment. In the illustrated embodiment, probe guidance system 400 includes multiple sensing systems 410a, 410b and additional sensing systems indicated by ellipsis 411, collectively referenced hereinafter as sensing systems 410. Probe guidance system 400 also includes a local network 420, an integrator system 450, a probe positioning system 430, a display 460 and a storage device 480. Although two sensing systems 410, one network 420, one integrator 450 and one storage device 480 are depicted in FIG. 4, in other embodiments a probe guidance system includes more or fewer such systems and components.

The integrator system 450 includes several processes. Integrator system 450 include processes 452a, 452b, and further processes indicated by ellipsis 453 (collectively referenced hereinafter as 3-D distribution processes 452), to generate 3-D distributions of properties based on data from corresponding sensing systems 410. Integrator system 450 includes merge process 454 to register and merge the 3-D distributions of properties received from processes 452. Integrator system 450 includes processes 456a, 456b (collectively referenced hereinafter as probe guidance processes 456), to provide information for guiding the probe in the patient. In the illustrated embodiment, the probe guidance processes 456 includes presentation process 456a to present probe position on a 3-D rendering of multiple distributions, and driving process 456b to control the movement and operation of the probe based on the probe position relative to the multiple 3-D distributions. Although two 3-D distribution processes 452, one presentation process 456a and one driving process 456b are depicted in FIG. 4, in other embodiments, one or more integrator systems include more or fewer such processes and communicate over a network, such as local network 420. For example, in some embodiments, the processes 452, 454, 456 in integrator 450 are performed on different computer devices, which communicate with each other, such as over local network 420.

Sensing systems 410 collect data about the distribution of one or more properties inside a volume in a patient, such as in the volume occupied by the heart. This data is communicated to integrator over local network 420 to be received by integrator 450. In other embodiments, these data are received in other manners, as described below. In some embodiments, the measurements are stored on data storage 480, or some other network storage device (not shown) connected to network 420. The 3-D distribution processes 452 generate 3-D distributions of one or more properties based on the data received from sensing systems 410. In some embodiments, the 3-D distributions are stored on data storage 480, or some other network storage device (not shown) connected to network 420.

The integrator system 450 receives real-time probe position data from probe positioning system 430. In some embodiments, the probe position data are stored on data storage device 480, or some other network storage device (not shown) connected to network 420.

Merge process 454 registers the 3-D distributions for an overlapping volume in the patient from processes 452 to the same frame of reference, such as the probe position frame of reference, and shows the current position of the catheter in that frame. In some embodiments, the merged 3-D distributions are stored on data storage 480, or some other network storage device (not shown) connected to network 420.

At least one of presentation process 456a and driving process 456b uses the merged 3-D and probe position data to guide the probe. Presentation process 456a presents the information as a 3-D rendering on display 460. In some embodiments, a human care-giver moves and operates the probe in response to the position of the probe displayed relative to the merged volume properties shown on display 460. Drive process 456b causes the probe to perform one or more functions, such as moving, taking samples or dispensing therapy (e.g., ablating nearby tissue) based on the position of the probe relative to the merged volume properties. In some embodiments, the drive process is automatic. In some embodiments a human care-giver uses the drive process 456a to move and operate the probe in response to the position of the probe displayed relative to the merged volume properties shown on display 460.

### 4. Method for Using Probe

Registering of an interventional electrophysiological catheter tip to a three dimensional (3-D) rendering of a single endocardial, surface based on CT slices collected during one mode of operation are described in Dong. and available in and available in Carto XP Electroanatomical Mapping System from BIOSENSE WEBSTER™, a JOHNSON & JOHNSON COMPANY, of New Brunswick, N.J.

According to embodiments of the invention, one or more different anatomical features from one or more different modes of operation for one or more sensing systems are used to guide the operation of a probe. Examples are given for an interventional electrophysiological catheter. In the illustrated embodiments, the anatomical features are determined and stored before the probe insertion; and the probe operation is based on the stored anatomical features. In other embodiments one or more of the anatomical features are determined in real time as the probe position is determined.

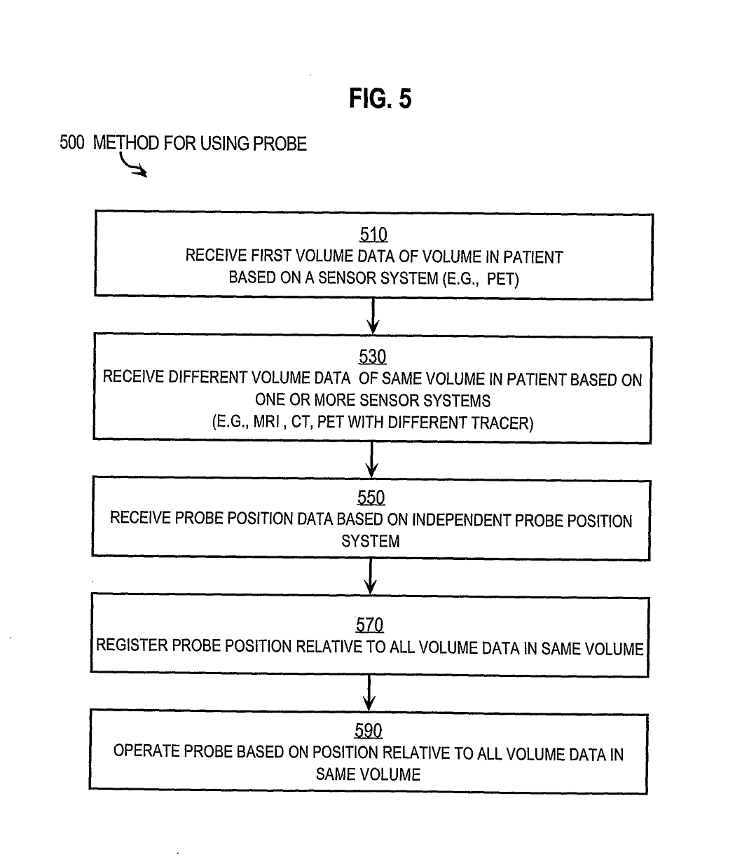

FIG. 5 is a flow diagram that illustrates at a high level a method for operating a probe based on one or more different anatomical structures from one or more different modes of operating one or more sensing systems, according to an embodiment. Although steps are shown in FIG. 5 in a particular order for purposes of illustration, in other embodiments the steps are performed in a different order or overlapping in time, or one or more steps are omitted, or the steps are changed in some combination of ways.

In step 510 first volume data is received. The first volume data is received at integrator 450 based on measurements of a particular volume in a particular patient from a first mode of operating a first sensing system, e.g., from sensing system 410a. For example, in some embodiments, the first volume data is the endocardial surface in the patient as determined from a series of CT scans with a particular contrast agent. As described in Dong, endocardial and intra-cardiac surfaces of the blood-to-tissue boundary were determined based on a single intensity range in the CT scans. In some embodiments, volume data includes segmented volumes associated with a particular kind of tissue or anatomical structure, such as ganglia, scar tissue, myocardial extent, among others, rather than just a surface having minimal thickness. In some embodiments, the volume data includes positions of multiple intensity ranges in the same set of scans. In some embodiments, other sensing systems are used to derive the 3-D distribution of other anatomical properties or other types of properties, as listed below.

In step 510, the 3-D distribution of properties in the volume of interest for the patient is received. Any method may be used to receive the first volume data, including, but not limited to, data stored within files or within a database accessible to the process, or from data included in a message sent to the system by another server or from a client process, such as a client process on a host operated by an human operator, output from a measurement device or input from a human operator in response to prompts from the process or independently of prompts, or some combination of methods. In some embodiments, step 510 includes operating a sensing system (such as the CT system) to acquire measurements, and deriving structural or process features from the data to produce 3-D mappings of those properties using process 452a. In some embodiments, step 510 includes placing markers in or on the patient that show up in the measurements by the sensing system and indicate the location of various landmarks in the patient.

Any spatially dependent property of the patient may be represented by the volume data, including structural, functional, metabolic, and histological properties. Examples of each are given in the following. In contrast to this approach, only a single endocardial surface feature is used in the process described by Dong.

Many complex arrhythmias like ventricular tachycardia (VT) are sustained by areas of scar tissue with slow electrical conduction. Myocardial scars are not a homogeneous structure but rather heterogeneous with strands of damaged, but viable tissue that serve as a critical limb in these arrhythmias. As these arrhythmias are often hemodynamically complex, it is impossible to define these areas electronically. Yet these areas often are the ablation target sites for successful treatment. With improving resolution of CT and MRI, which are already in the sub-millimeter range today (1 millimeter, mm, =10⁻³ meters), a detailed anatomic evaluation allows the determination of those target sites using imaging techniques. Mapping those target sites leads to shorter and more successful ablation procedures. Thus, with increasing resolution a rather histological evaluation of the tissue is possible, which enables more exact and successful treatment.

In the context of arrhythmia, functional imaging relates to the ability to assess cardiac contraction. Using different techniques including ultrasound, MRI, CT, optical mapping, Positron Emission Tomography (PET), nuclear medicine imaging systems, etc., it is possible to determine, for example, what area of the heart is contracting in what part of the cardiac cycle. As such, it is possible to determine the origin, course, and endpoint in three dimensions (3-D) of any given atrial or ventricular arrhythmia.

Metabolic imaging relies on differences in the cell metabolism from one area to the other, which can be helpful in determining abnormal areas. One measuring system for performing metabolic imaging includes Positron Emission Tomography (PET). With PET the uptake of sugar molecules (FDG tracer imaging) within a volume can be visualized, and is currently used to differentiate viable from not viable myocardium tissue. Current thresholds are <50% for non-viability and have been applied to determine the benefit of revascularization procedures. However, metabolic data has to be considered differently for electrophysiological applications, in which even cells with very low metabolism might be “alive enough” to participate in arrhythmias or to define sites suitable for treating with pacemakers. In some embodiments, PET deduced sugar uptake is mapped in 3-D, such as in FIG. 3B, with a more differentiated view than just the 50% cutoff, and received during step 510.

In some embodiments, a different PET tracer is employed during step 510 to produce volume data that indicates a different metabolic process. Using microPET (micron scale PET), for example, endothelin receptors antagonist can be imaged to detail specific disease processes (Johnstrom, P, Fryer T D, Richards H K, Barret O, Clark J C, Ohlstein E H, Pickard J D, Davenport AP, “In Vivo Imaging of Cardiovascular Endothelin Receptors Using the Novel Radiolabelled Antagonist [18F]-SB209670 and Positron Emission Tomography (microPET),” J Cardiovasc Pharmacol, v44, pp S34-S38, November 2004). Many cardiac and electrophysiological applications for PET are expected.

Molecular imaging techniques are among the most promising for many cardiac and most other medical applications. Differences in gene expressions, cell proteins, surface proteins etc. can be used to identify certain cell subgroups which are participating in genesis and maintenance of arrhythmia processes or other pathophysiological processes. Many different molecular imaging techniques are available; most currently use PET and MRI. An example is the family of connexin molecules which occur more frequently in the conduction system. Connexin 40 and 43 have been reported to be localized and likely contribute to initiation and maintenance of atrial flutter and atrial fibrillation (Kyungmoo, R. et al. “Pericarditis Induced Remodeling of Connexins 40 and 43 contributes to abnormal atrial conduction and arrhythmogenesis,” HeartRhythm. 205, 2:AB35-3, 2005). Similarly, connexin 43 and 45 may be associated with antifibrillatory properties (Li, et al. “Connexins 43 and 45 remodeling improves conduction safety in hibernating ground squirrel cittelus undulates,” HeartRhythm, 2:AB35-5, 2005). Changes of connexin 40 and 43 have been shown to occur with atrial fibrillation (Rasheda, A. et al., “Atrial fibrillation-induced connexin 43 redistribution in atria of the goat.” HeartRhythm. 2:AB48-3, 2005). Thus in some embodiments, the volume data received during step 510 includes a 3-D map of one such molecular indicator, such as an indicator for connexin 43.

In the future, maps containing the information where molecules, genes, proteins are located that participate in arrhythmias will allow new treatment strategies that implement molecularly guided ablation procedures. Even the imaging of an accessory bypass tract, foci for atrial arrhytmias like atrial fibrillation or ventricular arrhythmias may become possible. Many molecules like the connexins are under investigation to assess their role in atrial and ventricular arrhythinias. Nanoparticles can serve as modular platforms for a wide variety of highly sensitive and specific imaging agents. Bioluminescent imaging can provide in vivo analysis of multiple cellular and molecular events. Novel contrast agents can be conjugated with monoclonal antibodies for specific imaging. Endogenous and exogenous gene expression can be assessed and used for treatment and includes “indirect” reporter gene imaging, “direct” imaging of endogenous molecules, and “surrogate” and biomarker” imaging.

Histological imaging is used to produce volume data about cell types and populations in some embodiments. For example, the location and population of neurons are mapped as volume data by anatomic imaging of the ganglia and plexus, imaging of the adrenergic nervous system with tracers like 99 mTc-FBPBAT, iodine-123-MIBG and C-11-hydroxyephedrine and genetic signatures. More detailed imaging (e.g., micro-CT) should be able to provide quantitative analysis and histopathological insights. Thus in some embodiments, step 510 includes receiving volume data that indicates histological properties, such as these indicators of neuron concentration and location.

In step 530 second volume data is received. For example, sensing data from sensing system 410b is received and processed into volume data by process 452b. The second volume data is based on measurements of the same particular volume in the same particular patient from a second mode of operating the same or different sensing system. For example, in some embodiments, the second volume data is any of the volume data described above that is different from the actual volume data received in step 510. In various embodiments, the second volume data is based on measurements of the volume made with different measuring systems. For example, the first volume data is the endocardial boundary from CT system using one contrasting agent, and the second volume data is the uptake of sugar from a PET system with a particular tracer. It is anticipated that, often, the first volume entails some more general anatomic information, and the second dataset gives more detailed information about a specific subgroup like scar, nerve endings, etc.

In some embodiments, the second volume data is based on measurements of the volume made with the same measuring system, but with different variable components. For example, in some embodiments, both the first volume data and the second volume data are based on measurements from a CT system, but using different contrasting agents or taken at different times after the introduction of the contrasting agent. For example, one use for such embodiments in the treatment of arrhythmia is due to the fact that, after about five minutes, certain contrasting agents have washed in and washed out at different rates where there is scar tissue compared to where there is healthy tissue. Thus the first volume data shows the endocardial surface based on measurements shortly after the introduction of the contrasting agent, and the second volume data shows the location of scars within the myocardium based on measurements from the same sensing system about five to twenty-five minutes later.

In some embodiments that use novel volume data in step 510, step 530 is omitted. For example, in some embodiments, during step 510 volume data is received based on a PET system or other nuclear medicine technique. Such PET data and other nuclear medicine imaging techniques are not currently used to guide interventional electrophysiological procedures, and step 510 is sufficient to provide novelty for some embodiments. In other such embodiments, multiple anatomical features based on different intensity boundaries are included in the same volume data from a single mode of operating a single sensing system.

In some embodiments, additional volume data is received besides the first volume data and the second volume data. In various embodiments, the number of volume data sets received varies. Any volume data that provides additional structural, functional, metabolic, genetic and histological properties not already provided by other volume data is advantageously received in one or more other steps (not shown), which are analogous to step 530.

In step 550, probe position data is received based on an independent probe positioning system, e.g., from system 430. Any method known in the art may be used to position the probe, including, but not limited to, those based on electrical signals, fields and gradients, and those based on ultrasound. In an illustrated embodiment, an interventional electrophysiological catheter is positioned based on the attenuated signal received at the catheter from three electromagnetic coils embedded in a location pad beneath the patient.

In some embodiments, step 550 includes determining a simplified 3-D outline for a cavity by moving the probe to the boundaries of the cavity and determining the position of the probe at the boundary with the probe positioning system.

In step 570, information from the two or more measuring systems is merged, that is not merged in previous systems. Any method may be used to integrate the information from the probe positioning system and the one or more instances of volume data for an overlapping volume. For example, in various embodiments, a landmark registration or surface registration or both or other registration methods are employed for each volume data. Step 570 includes fusion of single or multiple volume data sets, e.g., anatomical, functional, metabolic or histological images, with the probe position data.

In step 590, the probe is guided based on the position of the probe relative to the first volume data and, if available, the second and additional volume data. For example, step 590 includes using the fused multiple images to facilitate complex ablation procedures. Scar-mediated VT is frequently impossible to map with electrophysiological measurements alone, and has to be treated based on anatomic considerations. For example, scar-mediated VT is treated by forming continuous ablation lesions from the scar center a) to healthy tissue, or b) to an adjacent scar or anatomic border, or c) by creating a tangential ablation lesion along the rim of the scar to interrupt the exit sites of VT. In some embodiments, the integration of images (MRI, CT, others) provide not only the endocardial shell (surface rendering) but also provide information about more of the myocardium, such as wall thickness, scar location, size, extent and transmurality (Kim, R J, Fieno D S, Parrish T B, Harris K, Chen E L, Simonetti O, Bundy J, Finn J P, Klocke F J, Judd R M, “Relationship of MRI delayed contrast enhancement to irreversible injury, infarct age, and contractile function,” Circulation, 9; v100 #19, pp 1992-2002, November 1999). As described above for CT systems, with some imaging protocols the scar-enhancement occurs at a time when the healthy myocardium does not show sufficient enhancement anymore to visualize scar and healthy myocardium at the same time. In such embodiments, two or more datasets acquired from the same system at different times or other dataset acquire specifically for each of the substrates are fused in step 570 to allow the full display of the arrhythmia substrate to guide the catheter during step 590.

Some VTs can not be successfully approached with an endocardial ablation and require mapping from the epicardium. This is associated with a significant risk of damaging the epicardial coronary arteries causing myocardial infarctions (D'Avila, A, Gutierrez P, Scanavacca M, Reddy V, Lustgarten D L, Sosa E, Ramires J A, “Effects of radiofrequency pulses delivered in the vicinity of the coronary arteries: implications for nonsurgical transthoracic epicardial catheter ablation to treat ventricular tachycardia,” Pacing Clin Electrophysiol. v25 #10, pp 1488-95, October 2002). Additionally, epicardial fat can simulate myocardial scar in electrophysiological measurements by demonstrating a decreased epicardial electrocardiogram (ECG) amplitude (Dixit, S, Narula N, Callans D J, Marchlinski F E, “Electroanatomic mapping of human heart: epicardial fat can mimic scar,” J Cardiovasc Electrophysiol. v14, #10, p 1128, 2003). Volume imaging as used for example with MRI or CT is able to visualize the anatomy, which is important to guide epicardial ablations. As such, in some embodiments, a single mode of operating one of these sensing systems shows the location of multiple structures, such as coronary arteries, epicardial fat, neuronal and related structures. Separate imaging modes are used for relevant structures (such as mentioned above) which cannot be adequately visualized with a single operating mode. All these are considered in step 590 to modify the ablation strategies. Improvement of the spatial resolution enables visualization of the heterogeneous histology of the scar; thus, anatomic correlates for areas of slow conduction, dense scar and viable myocardium are fused in some embodiments for an even more detailed ablation target map (Brunckhorst, C B, Delacretaz E, Soejima K, Jackman W M, Nakagawa H, Kuck K H, Ben-Haim S A, Seifert B, Stevenson W G. “Ventricular mapping during atrial and right ventricular pacing: relation of electrogram parameters to ventricular tachycardia reentry circuits after myocardial infarction,” J Interv Card Electrophysiol. v11 #3, pp 183-91, December 2004). In some embodiments, the volume data and ablation targets are selected to modify the nerve structures along the cervical and thoracic course of the catheter, as well as the insertion at the heart, to modify the sympathetic and parasympathetic input and modulation of the heart caused by such nerve structures.

Only one case report has described the use of CT and PET (Bello, D, Kipper S, Valderrabano M, Shivkumar K., “Catheter ablation of ventricular tachycardia guided by contrast-enhanced cardiac computed tomography,” Heart Rhythm, v1 #4, pp 490-2, October 2004). But this report used CT and PET only separately to determine the static location of a scar in the myocardium and did not present an integrated image to an electrophysiologist, either before or during an interventional electropysiological procedure. Thus this case does not determine probe position relative to the integrated CT and PET volume data.

In some embodiments, functional mapping is superimposed on an anatomic map to define the origin, exit points or “bottle necks” of an arrhythmia during step 570, which is used in some embodiments to determine a priority ablation site, to which the catheter is guided in step 590. For example, echo cardiograms have been used to determine the location of accessory pathways. With technological development, other technologies such as CT, MRI, optical mapping and others are anticipated to have the temporal resolution to provide functional guidance for targeted ablations.

In some embodiments, the targets are identified on the integrated volume data, and a probe, such as an electrophysiological catheter, is driven under automatic machine control to the site during step 590. For example, remote catheter control is performed with robots and other remote steering devices in two commercial remote catheter manipulation systems. Such systems are either commercially available already (Stereotaxis, see Ernst S, Ouyang F, Linder C, Hertting K, Stahl F, Chun J, Hachiya H, Bansch D, Antz M, Kuck K H. “Initial experience with remote catheter ablation using a novel magnetic navigation system: magnetic remote catheter ablation,” Circulation, Epub, v109, #12, pp 1472-5, March 2004) or on the horizon (see domain hansenmedical.com at directory news at file 050504-hrs. aspx on teh World Wide Web, www).

In some embodiments, the information is displayed in a 3-D rendering during step 590 for use by an electrophysiologist during a manual interventional electrophysiological procedure. For example, in various embodiments, the information is included with probe position either in a map with color coding representing the time lines for contraction, or shown on moving heart models, or both.

### 5. Computer Hardware Overview

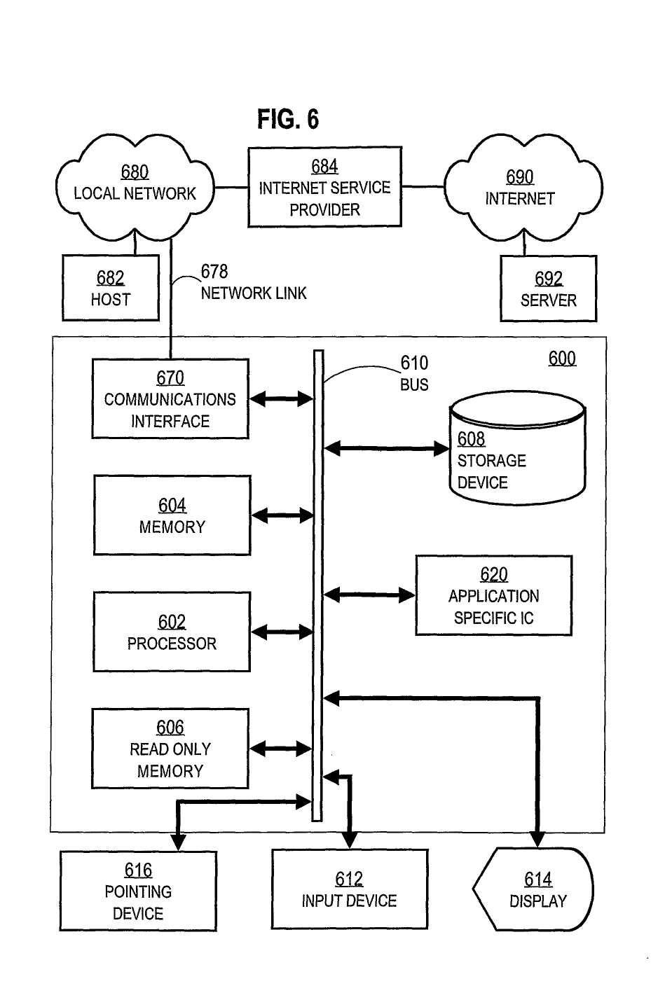

FIG. 6 is a block diagram that illustrates a computer system 600 upon which an embodiment of the invention may be implemented. Computer system 600 includes a communication mechanism such as a bus 610 for passing information between other internal and external components of the computer system 600. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). A sequence of binary digits constitutes digital data that is used to represent a number or code for a character. A bus 610 includes many parallel conductors of information so that information is transferred quickly among devices coupled to the bus 610. One or more processors 602 for processing information are coupled with the bus 610. A processor 602 performs a set of operations on information. The set of operations include bringing information in from the bus 610 and placing information on the bus 610. The set of operations also typically include comparing two or more units of information, shifting positions of units of information, and combining two or more units of information, such as by addition or multiplication. A sequence of operations to be executed by the processor 602 constitute computer instructions.

Computer system 600 also includes a memory 604 coupled to bus 610. The memory 604, such as a random access memory (RAM) or other dynamic storage device, stores information including computer instructions. Dynamic memory allows information stored therein to be changed by the computer system 600. RAM allows a unit of information stored at a location called a memory address to be stored and retrieved independently of information at neighboring addresses. The memory 604 is also used by the processor 602 to store temporary values during execution of computer instructions. The computer system 600 also includes a read only memory (ROM) 606 or other static storage device coupled to the bus 610 for storing static information, including instructions, that is not changed by the computer system 600. Also coupled to bus 610 is a non-volatile (persistent) storage device 608, such as a magnetic disk or optical disk, for storing information, including instructions, that persists even when the computer system 600 is turned off or otherwise loses power.

Information, including instructions, is provided to the bus 610 for use by the processor from an external input device 612, such as a keyboard containing alphanumeric keys operated by a human user, or a sensor. A sensor detects conditions in its vicinity and transforms those detections into signals compatible with the signals used to represent information in computer system 600. Other external devices coupled to bus 610, used primarily for interacting with humans, include a display device 614, such as a cathode ray tube (CRT) or a liquid crystal display (LCD), for presenting images, and a pointing device 616, such as a mouse or a trackball or cursor direction keys, for controlling a position of a small cursor image presented on the display 614 and issuing commands associated with graphical elements presented on the display 614.

In the illustrated embodiment, special purpose hardware, such as an application specific integrated circuit (IC) 620, is coupled to bus 610. The special purpose hardware is configured to perform operations not performed by processor 602 quickly enough for special purposes. Examples of application specific ICs include graphics accelerator cards for generating images for display 614, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 600 also includes one or more instances of a communications interface 670 coupled to bus 610. Communication interface 670 provides a two-way communication coupling to a variety of external devices that operate with their own processors, such as printers, scanners and external disks. In general the coupling is with a network link 678 that is connected to a local network 680 to which a variety of external devices with their own processors are connected. For example, communication interface 670 may be a parallel port or a serial port or a universal serial bus (USB) port on a personal computer. In some embodiments, communications interface 670 is an integrated services digital network (ISDN) card or a digital subscriber line (DSL) card or a telephone modem that provides an information communication connection to a corresponding type of telephone line. In some embodiments, a communication interface 670 is a cable modem that converts signals on bus 610 into signals for a communication connection over a coaxial cable or into optical signals for a communication connection over a fiber optic cable. As another example, communications interface 670 may be a local area network (LAN) card to provide a data communication connection to a compatible LAN, such as Ethernet. Wireless links may also be implemented. For wireless links, the communications interface 670 sends and receives electrical, acoustic or electromagnetic signals, including infrared and optical signals, that carry information streams, such as digital data. Such signals are examples of carrier waves.

The term computer-readable medium is used herein to refer to any medium that participates in providing information to processor 602, including instructions for execution. Such a medium may take many forms, including, but not limited to, non-volatile media, volatile media and transmission media. Non-volatile media include, for example, optical or magnetic disks, such as storage device 608. Volatile media include, for example, dynamic memory 604. Transmission media include, for example, coaxial cables, copper wire, fiber optic cables, and waves that travel through space without wires or cables, such as acoustic waves and electromagnetic waves, including radio, optical and infrared waves. Signals that are transmitted over transmission media are herein called carrier waves.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read.

Network link 678 typically provides information communication through one or more networks to other devices that use or process the information. For example, network link 678 may provide a connection through local network 680 to a host computer 682 or to equipment 684 operated by an Internet Service Provider (ISP). ISP equipment 684 in turn provides data communication services through the public, world-wide packet-switching communication network of networks now commonly referred to as the Internet 690. A computer called a server 692 connected to the Internet provides a service in response to information received over the Internet. For example, server 692 provides information representing video data for presentation at display 614.

The invention is related to the use of computer system 600 for implementing the techniques described herein. According to one embodiment of the invention, those techniques are performed by computer system 600 in response to processor 602 executing one or more sequences of one or more instructions contained in memory 604. Such instructions, also called software and program code, may be read into memory 604 from another computer-readable medium such as storage device 608. Execution of the sequences of instructions contained in memory 604 causes processor 602 to perform the method steps described herein. In alternative embodiments, hardware, such as application specific integrated circuit 620, may be used in place of or in combination with software to implement the invention. Thus, embodiments of the invention are not limited to any specific combination of hardware and software.

The signals transmitted over network link 678 and other networks through communications interface 670, which carry information to and from computer system 600, are exemplary forms of carrier waves. Computer system 600 can send and receive information, including program code, through the networks 680, 690 among others, through network link 678 and communications interface 670. In an example using the Internet 690, a server 692 transmits program code for a particular application, requested by a message sent from computer 600, through Internet 690, ISP equipment 684, local network 680 and communications interface 670. The received code may be executed by processor 602 as it is received, or may be stored in storage device 608 or other non-volatile storage for later execution, or both. In this manner, computer system 600 may obtain application program code in the form of a carrier wave.

Various forms of computer readable media may be involved in carrying one or more sequence of instructions or data or both to processor 602 for execution. For example, instructions and data may initially be carried on a magnetic disk of a remote computer such as host 682. The remote computer loads the instructions and data into its dynamic memory and sends the instructions and data over a telephone line using a modem. A modem local to the computer system 600 receives the instructions and data on a telephone line and uses an infra-red transmitter to convert the instructions and data to an infra-red signal, a carrier wave serving as the network link 678. An infrared detector serving as communications interface 670 receives the instructions and data carried in the infrared signal and places information representing the instructions and data onto bus 610. Bus 610 carries the information to memory 604 from which processor 602 retrieves and executes the instructions using some of the data sent with the instructions. The instructions and data received in memory 604 may optionally be stored on storage device 608, either before or after execution by the processor 602.

### 6. Extensions and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

What is claimed is:

1. A method for using a probe in a patient for a medical procedure, comprising the steps of:
receiving first volume data based on measurements of a particular volume in a particular patient from a first mode of operating a first sensing system;
receiving different second volume data based on measurements of the particular volume in the particular patient from a second mode of operating a second sensing system;
receiving probe position data that indicates a position of a probe inserted in the particular patient based on a probe positioning system independent of the first sensing system and the second sensing system;
determining probe position relative to the first volume data and the second volume data; and
operating the probe based on the probe position relative to the first volume data and the second volume data.

2. A method as recited in claim 1, said step of receiving first volume data further comprising receiving anatomical feature data that indicates the three dimensional extent of an anatomical feature based on measurements of the particular patient from the first sensing system.

3. A method as recited in claim 2, wherein the anatomical feature is a structure associated with arrhythmia.

4. A method as recited in claim 1, said step of receiving second volume data further comprising receiving physiological process data that indicates the three dimensional extent of a physiological process based on measurements of the particular patient from the second sensing system.

5. A method as recited in claim 2, wherein the physiological process is a process associated with arrhythmia.

6. A method as recited in claim 1, wherein:
the first sensing system is the same as the second sensing system; and
said step of receiving different second volume data from a second mode further comprising receiving the different second volume data based on measurements of the particular volume from operating the first sensing system at a different time.

7. A method as recited in claim 1, wherein:
the first sensing system is the same as the second sensing system; and
said step of receiving different second volume data from a second mode further comprising receiving the different second volume data based on measurements of the particular volume from operating the first sensing system using a different variable component for the system.

8. A method as recited in claim 7, wherein the variable component is at least one of an energy level for the system, a contrasting agent for the system, and a radioactively tagged tracer for the system.

9. A method as recited in claim 1, wherein said first sensing system and said second sensing system are selected from a group of sensing systems comprising a bi-planar Roentgen fluoroscopy system, a three dimensional (3-D) ultrasound reflectivity system, a computer tomography of Roentgen transmissivity (CT) system, a finer scale CT system, a micrometer-scale CT (micro-CT) system, a nuclear magnetic resonance (NMR) imaging (MRI) system, a Positron Emission Tomography (PET) system, a nuclear medicine camera and a molecular imaging system.

10. A method as recited in claim 1, said step of receiving probe position data further comprising receiving probe position data that indicates a position of a catheter that samples nearby tissue.

11. A method as recited in claim 10, said step of receiving probe position data further comprising receiving probe position data that indicates a position of an electrophysiological catheter that measures electrical impedance of nearby tissue.

12. A method as recited in claim 1, said step of receiving probe position data further comprising receiving probe position data that indicates a position of a catheter that applies therapy to nearby tissue.

13. A method as recited in claim 12, said step of receiving probe position data further comprising receiving probe position data that indicates a position of an ablation catheter that ablates nearby tissue when an energy source at the catheter is activated.

14. A method as recited in claim 1, said step of operating the probe further comprising changing a position of the probe.

15. A method as recited in claim 1, said step of operating the probe further comprising activating the probe to sample nearby tissue.

16. A method as recited in claim 1, said step of operating the probe further comprising activating the probe to apply therapy to nearby tissue.

17. A method as recited in claim 1, said step of determining probe position further comprising registering the first volume data with respect to the probe position data and registering the second volume data with respect to at least one of the probe position data and the first volume data.

18. A method as recited in claim 1, said step of operating the probe further comprising presenting a three dimensional graphical rendering of the first volume data in relation to the second volume data and the probe position data for viewing by a human medical care giver who determines how to operate the probe based on the three dimensional graphical rendering.

19. A method as recited in claim 1, wherein:
the method further comprises receiving additional volume data different from the first volume data and the second volume data based on measurements of the particular volume in the particular patient from different modes of operating one or more sensing systems;
said step of determining probe position further comprises determining probe position relative to the additional different volume data; and
said step of operating the probe further comprises operating the probe based on the probe position relative to the additional volume data.

20. A method for using a probe in a patient for a medical procedure, comprising the steps of:
receiving volume data based on measurements of a particular patient from a Positron Emission Tomography (PET) system;
receiving probe position data that indicates a position of a probe inserted in the particular patient based on a probe positioning system independent of the PET system;
determining probe position relative to the volume data; and
operating the probe based on the probe position relative to the volume data.

21. A method for using an electrophysiological catheter in a patient for treating arrhythmia, comprising the steps of:
receiving first volume data that indicates an endocardial surface of a chamber of a heart in a particular patient based on first measurements from a first sensing system;
receiving different volume data that indicates at least one of myocardial structure and myocardial process in a wall of the chamber of the heart of the particular patient based on different second measurements from a second sensing system that is one of the same sensing system and a different sensing system;
receiving catheter position data that indicates a position of an electrophysiological catheter inserted in the chamber of the heart of the particular patient based on an electrophysiological catheter positioning system independent of the first sensing system and the second sensing system;
determining catheter position relative to the endocardial surface and the myocardial structure; and
operating the catheter based on the catheter position relative to the endocardial surface and the myocardial structure to perform at least one of moving the catheter and ablating tissue in a vicinity of a tip of the electrophysiological catheter.

22. A computer-readable medium carrying one or more sequences of instructions for guiding a probe in a patient for a medical procedure, wherein execution of the one or more sequences of instructions by one or more processors causes the one or more processors to perform the steps of:
receiving first volume data based on measurements of a particular volume in a particular patient from a first mode of operating a first sensing system;
receiving different second volume data based on measurements of the particular volume in the particular patient from a second mode of operating a second sensing system;
receiving probe position data that indicates a position of a probe inserted in the particular patient based on a probe positioning system independent of the first sensing system and the second sensing system;
determining probe position relative to the first volume data and the second volume data; and
causing the probe to be operated based on the probe position relative to the first volume data and the second volume data.

23. A computer-readable medium as recited in claim 22, said step of causing the probe to be operated further comprising presenting a three dimensional graphical rendering of the first volume data in relation to the second volume data and the probe position data for viewing by a human medical care giver who determines how to operate the probe based on the three dimensional graphical rendering.

24. A computer-readable medium carrying one or more sequences of instructions for guiding a probe in a patient for a medical procedure, wherein execution of the one or more sequences of instructions by one or more processors causes the one or more processors to perform the steps of:
receiving volume data based on measurements of a particular patient from a Positron Emission Tomography (PET) system;
receiving probe position data that indicates a position of a probe inserted in the particular patient based on a probe positioning system independent of the PET system;
determining probe position relative to the volume data; and
causing the probe to be operated based on the probe position relative to the volume data.

25. A system for guiding a probe in a patient for a medical procedure, comprising:
means for receiving first volume data based on measurements of a particular volume in a particular patient from a first mode of operating a first sensing system;
means for receiving different second volume data based on measurements of the particular volume in the particular patient from a second mode of operating a second sensing system;
means for receiving probe position data that indicates a position of a probe inserted in the particular patient based on a probe positioning system independent of the first sensing system and the second sensing system;
means for determining probe position relative to the first volume data and the second volume data; and
means for operating the probe based on the probe position relative to the first volume data and the second volume data.

26. A system for guiding a probe in a patient for a medical procedure, comprising:
means for receiving volume data based on measurements of a particular patient from a Positron Emission Tomography (PET) system;
means for receiving probe position data that indicates a position of a probe inserted in the particular patient based on a probe positioning system independent of the PET system;
means for determining probe position relative to the volume data; and
means for operating the probe based on the probe position relative to the volume data.

27. A method for using a probe in a patient for a medical procedure, comprising the steps of:
receiving volume data based on measurements of a particular patient from a particular sensing system;
receiving probe position data that indicates a position of a probe inserted in the particular patient based on a probe positioning system independent of the particular sensing system;
determining a plurality of different boundaries representing a corresponding plurality of different anatomical features in the volume data;
determining probe position relative to the plurality of different boundaries; and
operating the probe based on the probe position relative to the plurality of different boundaries.

28. A computer-readable medium carrying one or more sequences of instructions for guiding a probe in a patient for a medical procedure, wherein execution of the one or more sequences of instructions by one or more processors causes the one or more processors to perform the steps of:
receiving volume data based on measurements of a particular patient from a particular sensing system;
receiving probe position data that indicates a position of a probe inserted in the particular patient based on a probe positioning system independent of the particular sensing system;
determining a plurality of different boundaries representing a corresponding plurality of different anatomical features in the volume data;
determining probe position relative to the plurality of different boundaries; and
causing the probe to be operated based on the probe position relative to the plurality of different boundaries.
